(19) United States
(12) Patent Application Publication
(10) Pub. No.: US 2002/0173790 A1
(43) Pub. Date: Nov. 21, 2002

(54) Device of an anterior cervical plate with a peg and screw

(76) Inventors: Han Chang, Seoul (KR); Byung-Kyu Yoon, Seoul (KR)

Correspondence Address: Fleshner & Kim, P O Box 221200, Chantilly, VA, 20153-1200, US

(21) Appl. No.: 10/130,627

(22) PCT Filed: Mar. 15, 2001
(86) PCT No.: PCT/KR01/00410

(30) Foreign Application Priority Data
Sep. 22, 2000 (KR) 2000-0055927
Mar. 8, 2001 (KR) 2001-0011976

(51) Int. Cl. A61F 2/30

(52) U.S. Cl. 606/61

(57) ABSTRACT

The invention relates to a plate which is used cervical anterior fixation surgery with a peg and screw. The cervical anterior fixation plate with a peg and screw not only prevents plate from being skewed, but defends from sinking a retractor hole which is made during a surgery. Heretofore, the cervical anterior fixation plate has peg and holes. Holes are including two for attaching pegs, and one for monitoring the movement of bone graft, and some for piercing screw. And the peg is composed of the head for attaching a plate and the stick for being inserted into a cervical vertebral body. Heretofore, the cervical anterior fixation plate removes the risk of injuring of nervous system and reduces the staff and the time for surgery.

### TECHNICAL FIELD

The present invention relates to the device of an anterior cervical plate with pegs and screws, and in particular, to anterior cervical insertion which is used for curing and fixation of cervical fracture, dislocation, tumor, kyphotic deformity, and cervical disc disease such as cervical hernial disc and cervical spondylosis myelopathy, in the field of orthopedics and neurosurgery.

### BACKGROUND ART

In general, most of diseases occurred in cervical spine are cured by cross fixation of each of cervical spine not to move, thus, for said fixation, a device for fixation which consists of plate and screw extruded from the plate has been used in common.

The screw of said fixation device is fixed upon cervical spine located on upper/lower limit of drawback. In general, curing is done by said fixation force of the screw and maintaining force of the plate which sustain the screw.

After incising drawback of patient and disclosing said cervical spine, in a state that the plate of said fixation device is faced on said drawback, the operation of said fixation device is performed. First, drill bit is inserted into screw hall of said fixation device, and screw insertion slot is formed by drill on said cervical spine at the same time. Next, while inserted from back of the plate, the screw of said fixation device is fixed upon cervical spine located on upper/lower limit of drawback.

For curing or elimination of injured cervical spine and disc among said cervical spine, A retractor is fitted on cervical spines located upper/lower limit of said injured cervical spine, and cervical spines are, using retractor, separated from said injured cervical spine. Then, operation of said cervical spine is performed.

Here, in a state that the plate faced on said drawback is fixed by assistant or by using subsidiary, the operation of said fixation device is performed by insertion of said screw.

According the above prior art, to insert screw in a state that the plate is faced on said drawback, separate assistant and another subsidiary are required for fixation of the plate, also additional time for utilization of said assistant and subsidiary is needed, thus the time of operation is increased.

Also, according the above prior art, to insert screw in a state that the plate is faced on said drawback, separation of said plate such as separation of said plate from drawback by external pressure and gyration of said plate by gyration/frictional-force of said screw, is occurred, therefore, accurate fixation of said plate is difficult, also freak of said drawback is induced in that said plate is fixed on separate position.

Also, to insert and operate the drill by using screw hall formed on said plate in a state that the plate is faced on said drawback, said plate is rotated from set-up position by frictional force between said drill and said screw hall of plate. To prevent it, said plate is fixed by additional component, thus, in the operation of cervical spine fixation, visibility of the operator is downscaled.

Also, according the prior art, for curing or elimination of injured cervical spine and disc, using said retractor, cervical spines or discs located upper/lower limit of said injured cervical spine are separated, then, the retractor hall formed on said upper/lower cervical spine to fix said retractor is caved after elimination of said retractor. Therefore, another damage on said cervical spine and disc arise, or another insertion should be interjected, thus efficiency of operation is decreased.

### DISCLOSURE OF INVENTION

In accordance with the present invention, in a state that upper/lower part of the plate faced on drawback is fixed, as screw is inserted through the back of said plate, fixation on said drawback is done. Thus, said plate is easily bonded and fixed on drawback.

Also, according to the present invention, retractor hall for using retractor formed on cervical spine of upper/lower part of drawback is sealed and filled, thus another operation for sealing of said retractor hall is not required, and caving of retractor hall is prevented.

Also, in accordance with the present invention, the device of an anterior cervical plate comprising drill bit inserted through screw hall of plate for forming screw insertion slot and peg which sustain against frictional force of said screw hall is provided.

To accomplish the above technical subject, according to the present invention, while peg extruded in anterior of plate is inserted into cervical spine located on upper/lower limit of drawback or bone graft, said plate is first fixed on said drawback. Then, said screw is inserted into each screw hall of said fixed plate, thus, said plate and cervical spine or disc is fixed.

Other feature of the present invention is that retractor hall formed on upper/lower cervical spine of drawback is sealed and filled by insertion of peg.

Another feature of the present invention is that drill inserted into screw hall of plate faced drawback and frictional force of said screw hall is sustained by fixation of said peg and cervical spine.

### BRIEF DESCRIPTION OF THE DRAWINGS

A better understanding of the present invention will be gained on reading the following detailed description and with reference to the appended figures that, only by way of not limiting example, illustrate some preferred embodiments thereof. In the drawings:

FIG. 1 is a view of fixation state of cervical spine in accordance with the present invention;

FIG. 2 is a perspective view of a device of an anterior cervical plate with a peg and screw in accordance with the present invention;

FIG. 3 is a front view of a device of an anterior cervical plate with a peg and screw in accordance with the present invention;

FIG. 4 is a front view of a peg in accordance with the present invention;

FIG. 5 is a cross-sectional view taken generally along the lines A-A of FIG. 2;

FIG. 6 is a cross-sectional view taken generally along the lines B-B of FIG. 3;

FIG. 7 is a cross-sectional view taken generally along the lines C-C of FIG. 3.

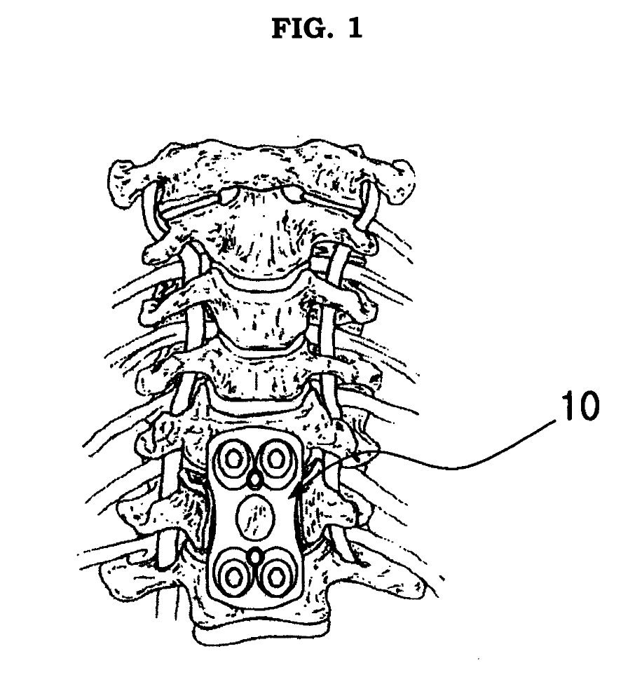

### BEST MODE FOR CARRYING OUT THE INVENTION

The device of anterior cervical plate(10) is attached to cervical spine located upper/lower limit of bone graft for cervical spine and bone graft to be cross-fixed after a cervical spine of patient is exchanged by bone graft.

Said device of anterior cervical plate(10) comprises plate(20) and screw(30) and peg(40).

Said plate(20) is formed with shape of rectangle plate. Observable hall(21) is formed in center of said plate(20) to observe for drawback.

Said screw(30) is inserted and screw-bonded into screw hall(31) formed roundly in 4 corner part of plate(20), and screw thread is formed outside thereof to be fixed into said screw hall(31) and cervical spine being rotate-inserted in one direction.

Here, it is preferred that screw hall(31) is fixed in plate(20), each screw hall(31) is formed individually in 4 corner part to sustain for each cervical spine.

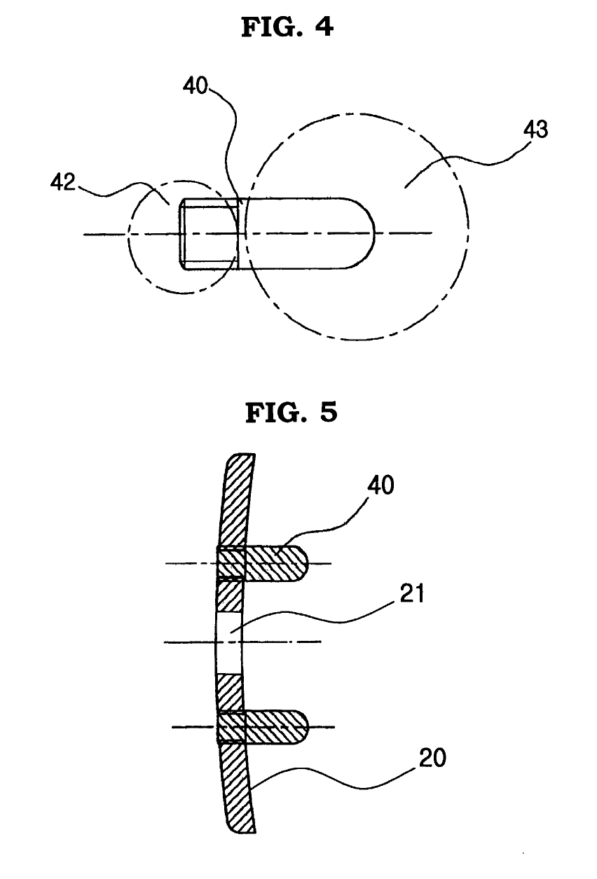

Said peg(40), as illustrated in FIG. 4, is barreled to be inserted into tapping hall(41) which is penetrated, circle-shaped and located in upper/lower part of observable hall(21) of plate(20). And said peg(40) consists of head part(42) where screw thread is formed with regular interval to be screw-bonded into screw thread formed inside of tapping hall(41) and sustaining bar(43) which is inserted into cervical spine and extruded apex of head part(42) and barrel-shaped.

Apex of sustaining bar(43) is formed by dome-shape. Thus, when sustaining bar(43) is inserted into bone graft and cervical spine, repulsive force against insertion is minimized.

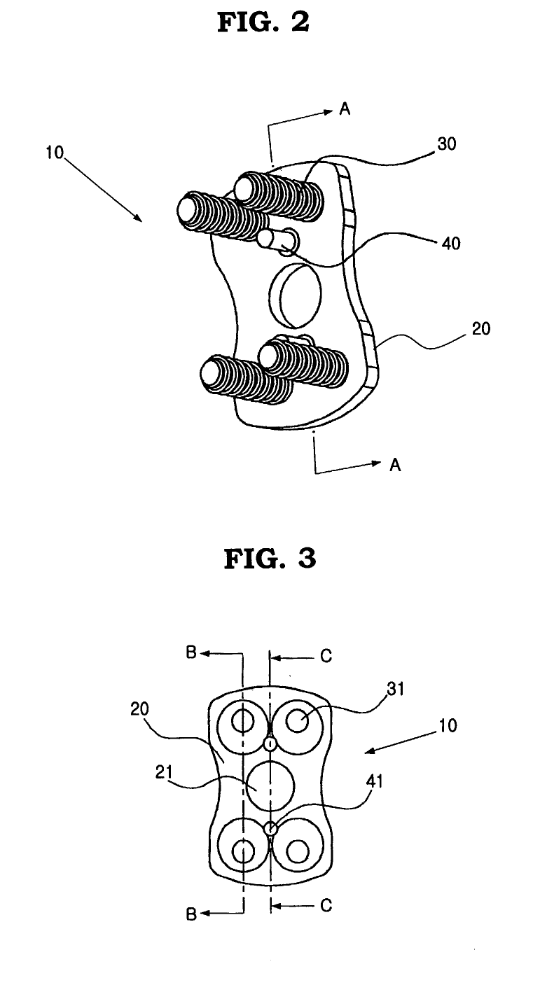

FIG. 5 is cross-sectional view which illustrate the state that peg(40) is fixed to plate(20) of device of anterior cervical plate(10) in FIG. 2. Peg(40) is inserted into tapping hall(41) in inside or outside of plate(20). Then, screw thread formed on head part(42) of peg(40) and screw thread formed on inside of tapping hall(41) are screw-bonded. Thus, peg(40) is combined with tapping hall(41) of plate(20).

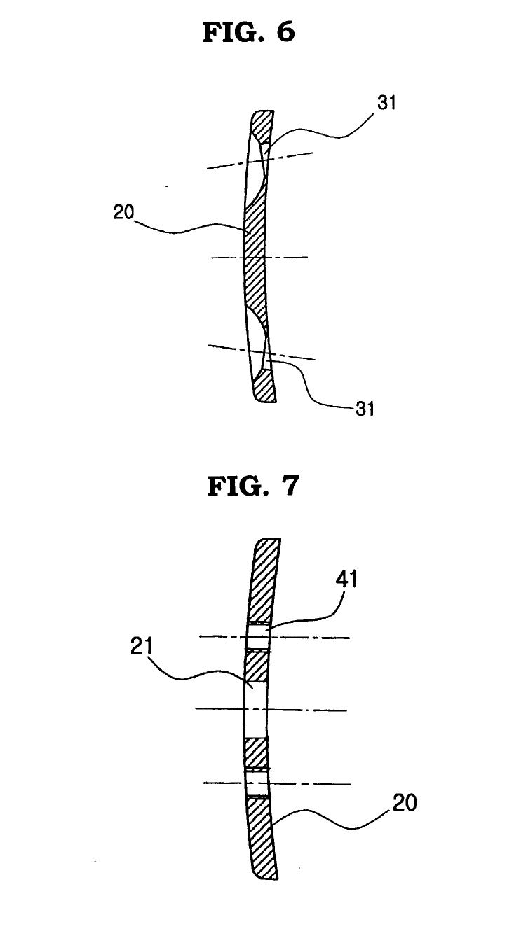

FIG. 6 is cross-sectional view of the part of screw hall(31) of plate(20) in FIG. 2. Inside of plate(20) is formed by sluggish curve according cervical spine screw hall(31) formed in 4 corner part of plate(20) is inclined for inside of plate(20), thus, screw(30) is actiniform extended and combined with plate(20).

FIG. 7 is cross-sectional view of the part of tapping hall(41) of plate(20) in FIG. 2. A pair of tapping hall(41) having side-by-side axis with pivot of plate(20) are formed in upper/lower part of observable hall(21) penetrated in center of plate(20).

Here, said tapping hall(41) is roundly penetrated, and screw thread is formed inside thereof to be combined with screw thread formed on outside of head part(42) of peg(40).

Also, said tapping hall(41) is aligned in upper/lower part of observable hall(21) so that it has diagonal sustaining force against external pressure applied on one limit of plate(20).

Next, operation process and function of an embodiment according the present invention is described.

In a case that a cervical spine or a disc among cervical spines or discs of patient is injured or crashed, said injured or crashed drawback of cervical spine is disclosed by incising outside tissue of cervical spine of said patient. Then, said injured or crashed cervical spine is operated to be sustained, or is replaced by bone graft.

Also, the device of anterior cervical plate(10) according to the present invention is operated to sustain said cervical spine or to fix bone graft replaced.

Here, operation of device of anterior cervical plate(11) for said injured, replaced cervical spine is to separate primarily cervical spines located in upper/lower part of said injured cervical spine from said injured cervical spine, or to separate using retractor for cervical spines located in upper/lower part of said injured cervical spine not to compress said injured cervical spine.

Here, retractor hall of circular shape is formed on upper/lower cervical spine of said injured cervical spine, and, screw of said retractor is inserted into said retractor hall. Then, said upper/lower cervical spine is separated from injured cervical spine by outthrust.

In a state that said upper/lower cervical spine is separated from injured cervical spine, peg(40) extruded to back of plate (20) is inserted into retractor hall so that said plate(20) is fixed first.

Then, 4 screw(30) is combined with each screw hall(31) of said fixed plate(20).

Here, said screw hall(31) is formed by drill on said upper/lower cervical spine of drawback. Then, frictional force of drill bit and screw hall(31) is sustained by insertion of peg into cervical spine.

Plate(20) fully sustain rotation pressure which arise when screw(30) rotates, because peg(40) is inserted into retractor hall not to rotate and screw(30) is inserted.

It is preferred that peg(40) should be inserted to cervical spine on which retractor hall is formed, in a state that head part(42) of peg(40) is inserted into tapping hall(41) in anterior of plate(20).

Also, peg(40) inserted into retractor hall sustain the pressure applied by cervical spine to retractor hall and external pressure of human tissues. Thus, abnormal state such as caving of retractor hall is obviated.

When gap of retractor hall formed in upper/lower part of injured cervical spine and gap of said a pair of peg(40) is different, plate(20) could be fixed in a state that a peg(40) of a pair of peg(40) is deviated from tapping hall(41) of plate(20) and said peg(40) is inserted into retractor hall formed on a cervical spine among said retractor hall.

Here, it is preferred that diameter of sustaining bar(43) of peg(40) is formed to be same as diameter of retractor hall formed on cervical spines for emplacement of retractor.

Thus, operation of cervical spine fixation is performed in a way that screw is inserted through screw hall(31) in the back of plate in a state that plate is incorporated with drawback by peg(40).

From the above description of the invention, those skilled in the art will perceive improvements, changes and modifications. Such improvements, changes and modifications within the skill of the art are intended to be covered by the appended claims.

### INDUSTRIAL APPLICABILITY

According to the present invention, when operation of cervical spine fixation is performed, plate is fixed by peg on regular position for drawback of cervical spine. Thus, separate assistant and another subsidiary are not to be required, also, said operation of cervical spine fixation is easily performed.

Other effect according to the present invention is that rotation of plate, resulted from frictional force by rotation of screw, is prevented by insertion/fixation force of peg, when screw is inserted into screw hall of plate.

Also, according to the present invention, when operation of cervical spine fixation is performed, retractor hall to be formed for adjustment of gap among said cervical spines is sealed and filled by insertion of peg. Thus, caving of retractor hall is prevented, and another operation for sealing of said retractor hall is not required. Therefore, efficiency of operation is increased.

What is claimed is:

1. In the device of an anterior cervical plate comprising a plate to be faced on the part of cervical spine fixation; and screw to be inserted and fixed into said cervical spine and plate,
The device of an anterior cervical plate with pegs and screws wherein a pair of pegs extrude and incorporated in upper/lower part of where plate and cervical spine are faced together.

2. The device according to claim 1, wherein head part with a screw thread is formed in said pegs, and a screw thread is formed on inside of the tapping hall of said plate with which said peg is incorporated, and said peg is screw-bonded into the tapping hall of said plate to be attachable and detachable.

3. The device according to claim 1, wherein said peg is inserted into two of retractor halls formed for adjustment of gap of cervical spines.
